(19) United States
(12) Patent Application Publication
(10) Pub. No.: US 2014/0073900 A1
(43) Pub. Date: Mar. 13, 2014

(54) SYSTEM AND METHOD FOR MEASURING CARDIAC OUTPUT

(71) Applicants: Lockett Wood, Lyons, CO (US); Bo Chen, Louisville, CO (US)

(72) Inventors: Lockett Wood, Lyons, CO (US); Bo Chen, Louisville, CO (US)

(73) Assignee: NELLCOR PURITAN BENNETT LLC, Boulder, CO (US)

(21) Appl. No.: 13/613,305

(22) Filed: Sep. 13, 2012

(51) Int. Cl. A61B 6/00; A61B 5/1455

(52) U.S. Cl. 600/407

(57) ABSTRACT

Embodiments of the present disclosure relate to a system and method for non-invasively determining a cardiac output of a patient that may include a photoacoustic sensor and a determination of oxygen uptake of the patient. Specifically, a signal from a photoacoustic sensor may be used to determine a mixed venous and an arterial oxygen saturation of the patient. The parameters of mixed venous and arterial oxygen saturation in conjunction with oxygen uptake may be used to calculate cardiac output using the Fick method.

### BACKGROUND

The present disclosure relates generally to medical devices and, more particularly, to the use of medical devices to non-invasively measure cardiac output.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In the field of medicine, doctors often desire to monitor certain physiological characteristics of their patients. Accordingly, a wide variety of devices have been developed for monitoring many such characteristics of a patient. Such devices provide doctors and other healthcare personnel with the information they need to provide the best possible healthcare for their patients. As a result, such monitoring devices have become an indispensable part of modern medicine.

For example, clinicians may wish to monitor a patient's blood flow and blood oxygen saturation to assess cardiac function. In particular, clinicians may wish to monitor a patient's cardiac output. The determination of cardiac output may provide information useful for the diagnosis and treatment of various disease states or patient abnormalities. For example, in cases of pulmonary hypertension, a clinical response may include a decrease in cardiac output.

Accordingly, there are a variety of clinical techniques which may be used for analyzing cardiac output. The direct Fick method, which calculates cardiac output as the quotient of the oxygen uptake and the difference of the arterial and mixed venous oxygen content, is generally regarded as the standard technique for determining cardiac output. Although the direct Fick method may be highly accurate, it involves the use of catheters and may be invasive to a patient. Further, the techniques for measuring various parameters (e.g., oxygen uptake) may be technically demanding. Thus, the direct Fick method is rarely used in a clinical setting to determine cardiac output. Accordingly, it may be beneficial to develop systems and methods for non-invasively monitoring cardiac output using the direct Fick method.

### BRIEF DESCRIPTION OF THE DRAWINGS

Advantages of the disclosed techniques may become apparent upon reading the following detailed description and upon reference to the drawings in which:

FIG. 1 is a block diagram of a patient monitor and photoacoustic sensor in accordance with an embodiment;

FIG. 2 is a block diagram of a method of determining cardiac output in accordance with an embodiment;

FIG. 3 is a schematic view of a system, including the patient monitor and one or more photoacoustic sensors of FIG. 1, for determining cardiac output using an oxygen uptake estimation in accordance with an embodiment;

FIG. 4 is a flow diagram of a method of determining cardiac output using an oxygen uptake estimation using the system of FIG. 3 in accordance with an embodiment;

FIG. 5 is a block diagram of a system, including the patient monitor and one or more photoacoustic sensors of FIG. 1 and a gas analysis device, for determining cardiac output of a ventilated patient in accordance with an embodiment; and

FIG. 6 is a flow diagram of a method of determining cardiac output of a patient using the system of FIG. 5.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Current techniques for monitoring a patient's cardiac output using the direct Fick method may be complex and invasive. To implement the direct Fick method, the Fick equation may be used, in which:

$$CO = \frac{{V}_{{O}_{2}}}{{C}_{a} - {C}_{v}} \qquad ( 1 )$$

where CO=cardiac output, V_{O2}=oxygen uptake, Cₐ=oxygen content of arterial blood, and Cᵥ=oxygen content of mixed venous blood. To measure Cₐ and Cᵥ, a clinician may take blood samples, often via a catheter, to analyze the oxygen content. Specifically, the clinician may use a catheter to obtain mixed venous blood from the pulmonary artery of a patient, as the pulmonary artery may provide the best source of mixed venous blood. Alternatively, the oxygen content of mixed venous blood may be derived using samples from the femoral and jugular veins. The arterial blood may be sampled from the pulmonary vein, or any suitable peripheral artery. Oxygen uptake may be determined by measuring the inspired and expired oxygen concentrations, as well as the expired minute volume. However, measuring the inspired oxygen concentration may be difficult, and small errors in the value may lead to highly inaccurate calculations of oxygen uptake. Thus, it may be desirable to provide a system and method for non-invasively and accurately monitoring the cardiac output of a patient.

Accordingly, the disclosed embodiments include using photoacoustic spectroscopy to non-invasively determine the oxygen content of arterial and mixed venous blood of a patient. Photoacoustic spectroscopy involves emitting light into a tissue such that the emitted light is absorbed by certain components of the tissue and/or blood. Tissue and/or blood in an interrogated region may absorb the emitted light and generate kinetic energy, which results in pressure fluctuations at the interrogated region. The pressure fluctuations may be detected in the form of acoustic radiation (e.g., ultrasound) by a sensor (e.g., a photoacoustic transducer). As different absorbers and concentrations of absorbers at an interrogated region may have different absorption properties, the amplitude of the detected acoustic radiation may be correlated to a density or concentration of a particular absorber. As such, photoacoustic spectroscopy may be used to determine the oxygen saturation of an interrogated region. The oxygen content may then be calculated from the oxygen saturation.

Furthermore, the disclosed embodiments provide systems and methods for determining the oxygen uptake of a patient to facilitate the calculation of cardiac output. In certain embodiments, oxygen uptake estimate may be determined by using an equation that relates the body surface area of the patient to oxygen uptake. Additionally, or alternatively, oxygen uptake may be measured using a mass flow device. In one embodiment, the mass flow device may be in line with a ventilator circuit of a patient, and as such, oxygen uptake may be measured continuously. In another embodiment, the mass flow device may be used to measure oxygen uptake intermittently for a spontaneously breathing patient.

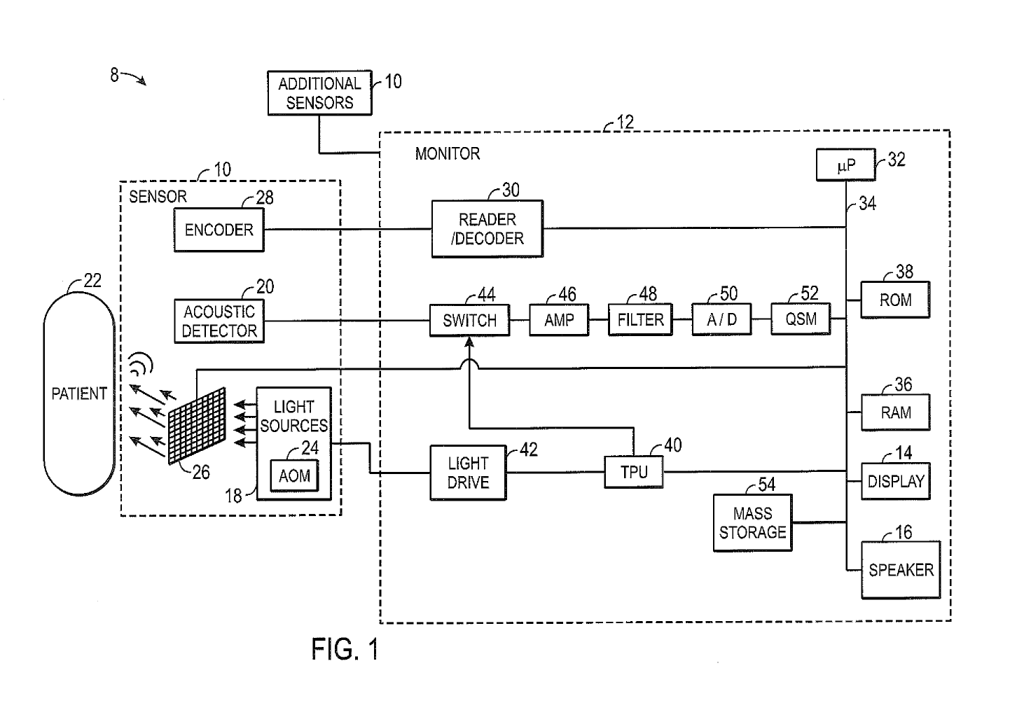

With the foregoing in mind, FIG. 1 depicts a photoacoustic spectroscopy system 8 that may be utilized in determining cardiac ouput. The system 8 includes a photoacoustic spectroscopy sensor 10 and a monitor 12. Some photoacoustic spectroscopy systems 8 may include one or more photoacoustic spectroscopy sensors 10, as illustrated in FIG. 1, to generate physiological signals for different regions of a patient. For example, in certain embodiments, a single sensor 10 may have sufficient penetration depth to generate physiological signals from deep vessels (e.g., pulmonary artery and/or pulmonary vein). In other embodiments, more than one (e.g., two sensors) sensor 10 may be used to monitor physiological parameters (e.g., oxygen saturation) of more superficial vessels (e.g., the jugular vein and the femoral vein).

The sensor 10 may emit spatially modulated light at certain wavelengths into a patient's tissue and may detect acoustic waves (e.g., ultrasound waves) generated in response to the emitted light. The monitor 12 may be capable of calculating physiological characteristics based on signals received from the sensor 10 that correspond to the detected acoustic waves. The monitor 12 may include a display 14 and/or a speaker 16 which may be used to convey information about the calculated physiological characteristics to a user. The sensor 10 may be communicatively coupled to the monitor 12 via a cable or, in some embodiments, via a wireless communication link.

In one embodiment, the sensor 10 may include a light source 18 and an acoustic detector 20, such as an ultrasound transducer. The present discussion generally describes the use of continuous wave (CW) light sources to facilitate explanation. However, it should be appreciated that the photoacoustic sensor 10 may also be adapted for use with other types of light sources, such as pulsed light sources, in other embodiments. In certain embodiments, the light source 18 may be associated with one or more optical fibers for conveying light from one or more light generating components to the tissue site.

The photoacoustic spectroscopy sensor 10 may include a light source 18 and an acoustic detector 20 that may be of any suitable type. For example, in one embodiment the light source 18 may be one, two, or more light emitting components (such as light emitting diodes) adapted to transmit light at one or more specified wavelengths. In certain embodiments, the light source 18 may include a laser diode or a vertical cavity surface emitting laser (VCSEL). The laser diode may be a tunable laser, such that a single diode may be tuned to various wavelengths corresponding to a number of different absorbers of interest in the tissue and blood. That is, the light may be any suitable wavelength or wavelengths (such as a wavelength between about 500 nm to about 1100 nm or between about 600 nm to about 900 nm) that is absorbed by a constituent of interest in the blood or tissue. For example, wavelengths between about 500 nm to about 600 nm, corresponding with green visible light, may be absorbed by deoxyhemoglobin and oxyhemoglobin. In other embodiments, red wavelengths (e.g., about 600 nm to about 700 nm) and infrared or near infrared wavelengths (e.g., about 800 nm to about 1100 nm) may be used. In one embodiment, the selected wavelengths of light may penetrate between 1 mm to 3 cm into the tissue of the patient 22. In certain embodiments, the selected wavelengths may penetrate through bone (e.g., the rib cage) of the patient 22.

One problem that may arise in photoacoustic spectroscopy may be attributed to the tendency of the emitted light to diffuse or scatter in the tissue of the patient 22. As a result, light emitted toward an internal structure or region, such as a blood vessel, may be diffused prior to reaching the region so that amount of light reaching the region is less than desired. Therefore, due to the diffusion of the light, less light may be available to be absorbed by the constituent of interest in the target region, thus reducing the ultrasonic waves generated at the target region of interest, such as a blood vessel. To increase the precision of the measurements, the emitted light may be focused on an internal region of interest by modulating the intensity and/or phase of the illuminating light.

Accordingly, an acousto-optic modulator (AOM) 24 may modulate the intensity of the emitted light, for example, by using LFM techniques. The emitted light may be intensity modulated by the AOM 24 or by changes in the driving current of the LED emitting the light. The intensity modulation may result in any suitable frequency, such as from 1 MHz to 10 MHz or more. Accordingly, in one embodiment, the light source 18 may emit LFM chirps at a frequency sweep range approximately from 1 MHz to 5 MHz. In another embodiment, the frequency sweep range may be of approximately 0.5 MHz to 10 MHz. The frequency of the emitted light may be increasing with time during the duration of the chirp. In certain embodiments, the chirp may last approximately 1 second or less and have an associated energy of a 10 mJ or less, such as between 1 μJ to 2 mJ, 1-5 mJ, 1-10 mJ. In such an embodiment, the limited duration of the light may prevent heating of the tissue while still emitting light of sufficient energy into the region of interest to generate the desired acoustic waves when absorbed by the constituent of interest.

Additionally, the light emitted by the light source 18 may be spatially modulated, such as via a modulator 26. For example, in one embodiment, the modulator 26 may be a spatial light modulator, such as a Holoeye® LC-R 2500 liquid crystal spatial light modulator. In one such embodiment, the spatial light modulator may have a resolution of 1024×768 pixels or any other suitable pixel resolution. During operation, the pixels of the modulator 26 may be divided into subgroups (such as square or rectangular subarrays or groupings of pixels) and the pixels within a subgroup may generally operate together. For example, the pixels of a modulator 26 may be generally divided into square arrays of 10×10, 20×20, 40×40, or 50×50 pixels. In one embodiment, each subgroup of pixels of the modulator 26 may be operated independently of the other subgroups. The pixels within a subgroup may be operated jointly (i.e., are on or off at the same time) though the subgroups themselves may be operated independently of one another. In this manner, each subgroup of pixels of the modulator 26 may be operated so as to introduce phase differences at different spatial locations within the emitted light. That is, the modulated light that has passed through one subgroup of pixels may be at one phase and that phase may be the same or different than the modulated light that has passed through other subgroups of pixels, i.e., some segments or portions of the modulated light wavefront may be ahead of or behind other portions of the wavefront. In one embodiment, the modulator 26 may be associated with additional optical components (e.g., lenses, reflectors, refraction gradients, polarizers, and so forth) through which the spatially modulated light passes before reaching the tissue of the patient 22.

In one example, the acoustic detector 20 may be one or more ultrasound transducers suitable for detecting ultrasound waves emanating from the tissue in response to the emitted light and for generating a respective optical or electrical signal in response to the ultrasound waves. For example, the acoustic detector 20 may be suitable for measuring the frequency and/or amplitude of the ultrasonic waves, the shape of the ultrasonic waves, and/or the time delay associated with the ultrasonic waves with respect to the light emission that generated the respective waves. In one embodiment an acoustic detector 20 may be an ultrasound transducer employing piezoelectric or capacitive elements to generate an electrical signal in response to acoustic energy emanating from the tissue of the patient 22, i.e., the transducer converts the acoustic energy into an electrical signal.

In one implementation, the acoustic detector 20 may be a low finesse Fabry-Perot interferometer mounted on an optical fiber. In such an embodiment, the incident acoustic waves emanating from the probed tissue modulate the thickness of a thin polymer film. This produces a corresponding intensity modulation of light reflected from the film. Accordingly, the acoustic waves are converted to optical information, which is transmitted through the optical fiber to an upstream optical detector, which may be any suitable detector. In some embodiments, a change in phase of the detected light may be detected via an appropriate interferometry device which generates an electrical signal that may be processed by the monitor 12. The use of a thin film as the acoustic detecting surface allows high sensitivity to be achieved, even for films of micrometer or tens of micrometers in thickness. In one embodiment, the thin film may be a 0.25 mm diameter disk of 50 micrometer thickness polyethylene terepthalate with an at least partially optically reflective (e.g., 40% reflective) aluminum coating on one side and a mirror reflective coating on the other (e.g., 100% reflective) that form the mirrors of the interferometer. The optical fiber may be any suitable fiber, such as a 50 micrometer core silica multimode fiber of numerical aperture 0.1 and an outer diameter of 0.25 mm.

The photoacoustic sensor 10 may include a memory or other data encoding component, depicted in FIG. 1 as an encoder 28. For example, the encoder 28 may be a solid state memory, a resistor, or combination of resistors and/or memory components that may be read or decoded by the monitor 12, such as via reader/decoder 30, to provide the monitor 12 with information about the attached sensor 10. For example, the encoder 28 may encode information about the sensor 10 or its components (such as information about the light source 18 and/or the acoustic detector 20). Such encoded information may include information about the configuration or location of photoacoustic sensor 10, information about the type of lights source(s) 18 present on the sensor 10, information about the wavelengths, light wave frequencies, chirp durations, and/or light wave energies which the light source(s) 18 are capable of emitting, information about the nature of the acoustic detector 20, and so forth. In certain embodiments, the information also includes a reference linear frequency modulation (LFM) chirp that was used to generate the actual LFM emitted light. This information may allow the monitor 12 to select appropriate algorithms and/or calibration coefficients for calculating the patient's physiological characteristics, such as the amount or concentration of a constituent of interest in a localized region, such as a blood vessel.

In one implementation, signals from the acoustic detector 20 (and decoded data from the encoder 28, if present) may be transmitted to the monitor 12. The monitor 12 may include data processing circuitry (such as one or more processors 32, application specific integrated circuits (ASICS), or so forth) coupled to an internal bus 34. Also connected to the bus 34 may be a RAM memory 36, a ROM memory 38, a speaker 16 and/or a display 14. In one embodiment, a time processing unit (TPU) 40 may provide timing control signals to light drive circuitry 42, which controls operation of the light source 18, such as to control when, for how long, and/or how frequently the light source 18 is activated, and if multiple light sources are used, the multiplexed timing for the different light sources.

The TPU 40 may also control or contribute to operation of the acoustic detector 20 such that timing information for data acquired using the acoustic detector 20 may be obtained. Such timing information may be used in interpreting the acoustic wave data and/or in generating physiological information of interest from such acoustic data. For example, the timing of the acoustic data acquired using the acoustic detector 20 may be associated with the light emission profile of the light source 18 during data acquisition. Likewise, in one embodiment, data acquisition by the acoustic detector 20 may be gated, such as via a switching circuit 44, to account for differing aspects of light emission. For example, operation of the switching circuit 44 may allow for separate or discrete acquisition of data that corresponds to different respective wavelengths of light emitted at different times.

The received signal from the acoustic detector 20 may be amplified (such as via amplifier 46), may be filtered (such as via filter 48), and/or may be digitized if initially analog (such as via an analog-to-digital converter 50). The digital data may be provided directly to the processor 32, may be stored in the RAM 36, and/or may be stored in a queued serial module (QSM) 52 prior to being downloaded to RAM 36 as QSM 52 fills up. In one embodiment, there may be separate, parallel paths for separate amplifiers, filters, and/or A/D converters provided for different respective light wavelengths or spectra used to generate the acoustic data.

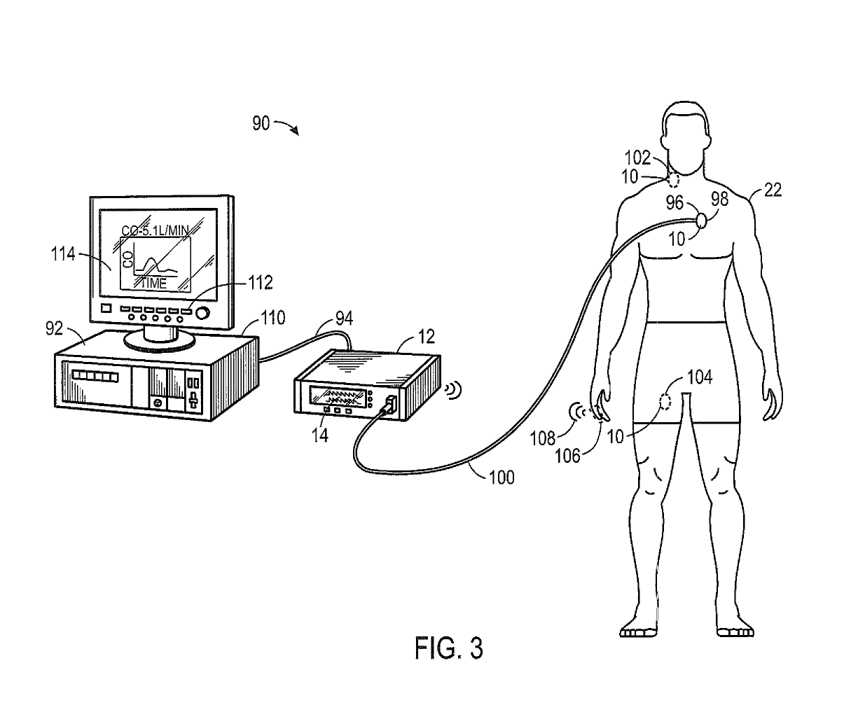

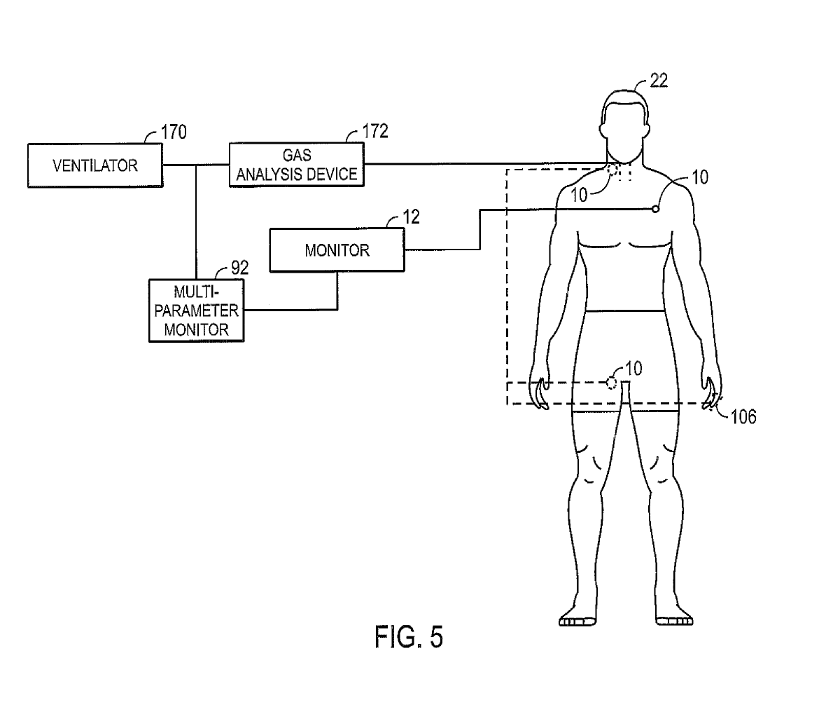

In certain embodiments, the data processing circuitry, such as processor 32, may perform a signal quality assessment on the digital data. For example, it may be desirable to use a single, transthoracic photoacoustic sensor 10 to directly obtain the arterial and mixed venous oxygen saturation measurements from the pulmonary vein and pulmonary artery, respectively. Signals generated by interrogating the pulmonary artery may yield more accurate measurements for the mixed venous oxygen saturation. However, the depth of penetration of the photoacoustic sensor 10 may not be sufficient to interrogate the pulmonary artery for certain patients 22. Accordingly, it may be desirable to compare the digital data received from the sensor 10 to a threshold value (e.g., a signal quality threshold value) to determine whether the penetration depth is sufficient for transthoracic measurements. Additionally, as will be described in more detail below with respect to FIGS. 3 and 5, multiple sensors 10 may be coupled to the monitor 12. As such, the processor 32 may compare the digital signals received from each sensor 10 to the threshold value and based at least in part upon the comparisons, select digital signals from one or more sensors 10 to further process and/or use in the calculation of one or more physiological characteristics (e.g., oxygen saturation).

The data processing circuitry, such as processor 32, may derive one or more physiological characteristics based on data generated by the photoacoustic sensor 10. For example, based at least in part upon data received from the acoustic detector 20, the processor 32 may calculate the amount or concentration of a constituent of interest in a localized region of tissue or blood using various algorithms. In certain embodiments, the processor 32 may calculate arterial and/or mixed venous oxygen saturation using signals obtained from one or more sensors 10. In one embodiment, the processor 32 may calculate both mixed venous and arterial oxygen saturation using signals obtained from a signal sensor 10. In certain embodiments, these algorithms may use coefficients, which may be empirically determined, that relate the detected acoustic waves generated in response to emitted light waves at a particular wavelength or wavelengths to a given concentration or quantity of a constituent of interest within a localized region.

In one embodiment, processor 32 may access and execute coded instructions, such as for implementing the algorithms discussed herein, from one or more storage components of the monitor 12, such as the RAM 36, the ROM 38, and/or a mass storage 54. Additionally, the RAM 36, ROM 38, and/or the mass storage 54 may serve as data repositories for information such as templates for LFM reference chirps, coefficient curves, and so forth. For example, code encoding executable algorithms may be stored in the ROM 38 or mass storage device 54 (such as a magnetic or solid state hard drive or memory or an optical disk or memory) and accessed and operated according to processor 32 instructions using stored data. Such algorithms, when executed and provided with data from the sensor 10, may calculate one or more physiological characteristics as discussed herein (such as the type, concentration, and/or amount of a constituent of interest). Once calculated, the physiological characteristics may be displayed on the display 14 for a caregiver to monitor or review. Additionally, the calculated physiological characteristics, such as the arterial and the mixed venous oxygen saturation values, may be sent to a multi-parameter monitor for further processing (e.g., for the calculation of cardiac output) and display. Alternatively, the processor 32 may use the algorithms to calculate the cardiac ouput and the cardiac ouput may be displayed on the display 14 of the monitor 12.

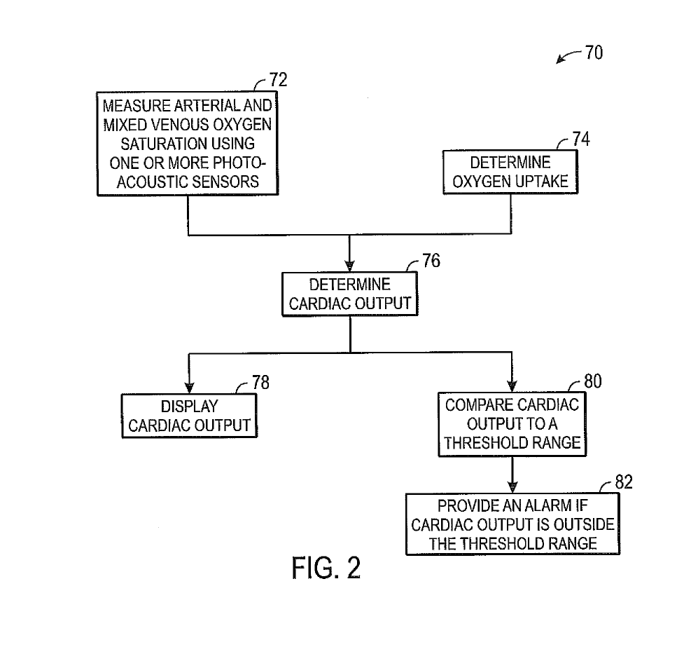

With the foregoing in mind, FIG. 2 illustrates a method 70 for monitoring the cardiac output of a patient using photoacoustic spectroscopy in accordance with some embodiments. The method 70 may be performed as an automated procedure by a system, as will be described in more detail below with respect to FIGS. 3 and 5. In addition, certain steps of the method 70 may be performed by a processor, or a processor-based device such as the monitor 12 that includes instructions for implementing certain steps of the method 70.

The method 70 may include non-invasively measuring the arterial and mixed venous oxygen saturation using one or more photoacoustic sensors 10 (block 72). In some embodiments, both the arterial and mixed venous oxygen saturation may be determined from a single transthoracic sensor 10. In other embodiments, signals from two or more photoacoustic sensors 10 may be used. For example, in one embodiment, the mixed venous saturation may be derived using signals from a first sensor 10 disposed about the jugular vein of the patient 22 and a second sensor 10 disposed about the femoral vein of the patient 22. Additionally, a third sensor 10, or alternatively, a pulse oximetry sensor, may be disposed about a peripheral artery of the patient 22 to obtain signals related to the arterial oxygen saturation. The monitor 12 may perform analysis of the one or more signals from the one or more photoacoustic sensors 10 and calculate the arterial and mixed venous oxygen saturation, as previously described.

The method 70 may also include determining oxygen uptake of the patient 22 (block 74). The determining of oxygen uptake (block 74) may be performed independent of measuring the arterial and mixed venous oxygen saturation (block 72). Additionally, in certain embodiments, oxygen uptake may be an estimate based upon the body surface area of the patient 22. In other embodiments, oxygen uptake may be derived using a mass flow device that analyzes the inhaled and end-tidal respiratory gases of the patient 22.

After the values for arterial and mixed oxygen saturation and oxygen uptake have been determined, cardiac output may be determined (block 76). In certain embodiments, the monitor 12 may receive the oxygen uptake parameter from the mass flow device or, alternatively, from another processor or processor-based monitor. As such, the monitor 12 may be configured to use the determined values to calculate cardiac output using the Fick method. In other embodiments, the method 70 may include another processor or processor-based device, such as a multi-parameter monitor, that may be configured to receive the determined values and may apply various algorithms to calculate cardiac output using the Fick method. For example, as discussed above, the oxygen content of arterial or mixed venous blood may be calculated using the respective oxygen saturation value. Specifically, in one embodiment, the oxygen content may be determined in accordance with the equation:

C=(H_{B}×k₁×S)/100 (2)

where C is the oxygen content of arterial or mixed venous blood (e.g., Cₐ or Cᵥ), k₁ is a coefficient, H_{B} is the hemoglobin, and S is the oxygen saturation of arterial or mixed venous blood (e.g., Sₐ or Sᵥ). To determine oxygen content for both arterial and mixed venous blood, Equation 2 may be applied once using the determined arterial oxygen saturation and once using the determined mixed venous oxygen saturation. The hemoglobin of the patient 22 may be readily measured using known techniques, such as supplying a small sample of blood taken via a finger prick to a hemoglobin photometer. In certain embodiments, the monitor 12 may be configured to calculate the oxygen content values. Accordingly, the values for arterial and mixed venous oxygen content and the value for oxygen uptake may then be applied to Equation 1 to determine cardiac output.

Furthermore, the method 70 may include displaying the cardiac ouput (block 78) on a display of the monitor 12 and/or a multi-parameter monitor for a user to monitor or review. Additionally, the processor-based device (e.g., monitor 12 and/or multi-parameter monitor) may compare the determined cardiac output to a threshold or threshold range (e.g., a low threshold and a high threshold) related to a normal and/or acceptable range of cardiac output values (block 80). In certain embodiments, a user may input parameters related to the patient 22, such as age, gender, and/or weight, to the processor-based device. The processor-based device may be configured to apply the parameters to various algorithms to determine the threshold or threshold range specific for the patient 22. Alternatively, the processor-based device may include various predetermined threshold ranges, and a user may simply select the appropriate range for each patient 22. Furthermore, the processor-based device may be configured to provide an alarm (block 82) to the user in response to determining that the cardiac output parameter is outside of the predetermined threshold range. The alarm may be a visual indication, such as an error message, a flashing light, or a change in color and/or size of the displayed cardiac output parameter. The alarm may also be an auditory indication, such as a beep.

As described above, the oxygen uptake may be determined using an estimation of the body surface area of the patient 22. In certain circumstances, it may be advantageous to use the body surface area estimation, as body surface area may be easily and non-invasively determined. A clinician may be provided with graphs and/or charts relating to height and weight, which may allow the clinician to readily determine the body surface area of the patient 22. Alternatively, the body surface area may be calculated using various algorithms (e.g., the Mosteller formula or the DuBois and DuBois formula). It is known that oxygen uptake is linearly related to body surface area, and as such, the oxygen uptake may be readily calculated using a known or empirically determined coefficient.

Accordingly, FIG. 3 illustrates an embodiment of a system 90 that may be operable to monitor the cardiac output of the patient 22 using a body surface area estimation to determine oxygen uptake. The system 90 includes one or more photoacoustic sensors 10, the monitor 12, and a multi-parameter monitor 92. The multi-parameter monitor may be communicatively coupled to the monitor 12 via a cable 94 connected to a sensor input port or a digital communication port. Alternatively, the monitor 12 and the multi-parameter monitor 92 may be configured to communicate wirelessly.

As previously described, based at least in part upon the received signals corresponding to the acoustic waves received by detector 20 of the sensor 10, the microprocessor 32 of the monitor 12 may calculate the oxygen saturation using various algorithms. In certain embodiments, the system 90 may include a single, transthoracic sensor 10 disposed about the pulmonary artery 96 and the pulmonary vein 98 of the patient 22 for generating signals corresponding to the mixed venous and arterial oxygen saturation, respectively. The transthoracic sensor 10 may be coupled to the monitor 12 via a cable 100. As described above, the microprocessor 32 may compare the signals received from the transthoracic sensor 10 to a threshold (e.g., signal quality threshold) and may determine whether the transthoracic sensor 10 has sufficient penetration depth for transthoracic measurements. Accordingly, the system 90 may additionally, or alternatively, include a sensor 10 disposed about the jugular vein 102 and a sensor 10 disposed about the femoral vein 104 for deriving the mixed venous oxygen saturation for circumstances in which the penetration depth is insufficient for transthoracic measurements. For illustration purposes, the jugular and the femoral sensor 10 are displayed with dashed lines to indicate that, in certain embodiments, the jugular and femoral sensors 10 may not be applied to the patient 22 and, in other embodiments, the jugular and femoral sensors 10 may be applied but not used (e.g., as backup in case the transthoracic sensor 10 is insufficient). Again, for illustration purposes, the jugular and femoral sensors 10 are depicted without cables 100. However, it should be appreciated that they may also be coupled to the monitor 12 via one or more cables 100. Further, for embodiments in which the transthoracic sensor 10 is insufficient, the system 90 may include an additional sensor 10 (not shown) or a pulse oximetry sensor 106 disposed about a peripheral artery (e.g., disposed about a finger of the patient 22) for determining the arterial oxygen saturation. The pulse oximetry sensor 106 may be communicatively coupled to the monitor 12 via a cable or wireless communication 108.

After determining the arterial and mixed venous oxygen saturation parameters, the monitor 12 may transmit the parameters to the multi-parameter monitor 92 for further analysis and calculation of cardiac output using the Fick method. The multi-parameter monitor 92 may include a processor 110 configured to execute code to perform the techniques as described herein. Alternatively, the processor 32 of the monitor 12 may process the determined parameters and calculate cardiac output using the Fick method. Accordingly, the processor 32, 110 may calculate the arterial and mixed venous oxygen content based in part upon the received arterial and mixed venous oxygen saturation parameters. The processor 32, 110 may apply various algorithms, such as Equation 2. These algorithms may employ certain coefficients, which may be empirically determined. The algorithms and coefficients may be stored in any suitable computer-readable storage medium and accessed and operating according to processor 32, 110 instructions.

Additionally, the processor 32, 110 may be configured to calculate body surface area and/or oxygen uptake using various algorithms based on clinician inputs entered via control inputs 112 on the monitor 92, a keyboard, or other device. For example, a clinician may input a patient's height, weight, and/or gender, which may be employed in various algorithms to determine body surface area and additionally, in certain embodiments, to determine body mass index (BMI). Alternatively, the clinician may input a determined body surface area of the patient 22 via the control inputs 112. Furthermore, the processor 32, 110 may be configured to apply various algorithms to calculate the oxygen uptake based on the determined body surface area. In one embodiment, the algorithms may employ certain coefficients, which may be empirically determined, that correspond to the BMI and/or gender of the patient 22. For example, certain patients 22, such as patients 22 with a BMI greater than 35 kg/m², or more specifically, male patients 22 with a BMI greater than 40 kg/m², may have a greater oxygen uptake than the determined oxygen uptake based on the body surface area estimate. Using the estimation of oxygen uptake may be advantageous as the system 90 may continuously monitor cardiac output for patients 22 on a mechanical ventilator, as well as spontaneously breathing patients 22. That is, the estimation of oxygen uptake does not require analyzing the inhaled and exhaled gas of the patient 22, which may be technically complex for the spontaneously breathing patient 22.

After determining the parameters for the Fick method, the processor 32, 110 may calculate and monitor cardiac output. Specifically, the processor 32, 110 may apply Equation 1 for determining cardiac output. To monitor cardiac output over time, a baseline measurement of cardiac output may be established by an independent measurement (e.g., blood samples taken to apply the direct Fick method) and may be used to calibrate cardiac output determined using body surface area. Specifically, the processor 32, 110 may determine a calibration coefficient for the estimation of oxygen uptake in accordance with the equation:

k₂=CO_{b}×(H_{B}×(S_{aO2}−S_{vO2}))_{b} (3)

where k₂ is the calibration coefficient, CO_{b} is the baseline cardiac output parameter, H_{B} is the baseline hemoglobin parameter, S_{aO2} is the baseline arterial oxygen saturation parameter, and S_{vO2} is the baseline mixed venous oxygen saturation parameter. The parameters for arterial and mixed venous oxygen saturation, and accordingly for arterial and mixed venous oxygen content, may fluctuate with time. As such, in one embodiment, processor 32, 110 may monitor cardiac output over time (e.g., a trend in cardiac output) in accordance with the equation:

$$CO ( t ) = \frac{{k}_{2}}{( {H}_{B} ( t ) \times ( {S}_{aO 2} ( t ) - {S}_{v O 2} ( t ) ) )} \qquad ( 4 )$$

where k₂ is the calibration coefficient, CO is cardiac output as a function of time, H_{B} is hemoglobin as a function of time, S_{aO2} is arterial oxygen saturation as a function of time, and S_{vO2} is mixed venous oxygen saturation as a function of time.

The multi-parameter monitor 92 may provide a display 114 to facilitate the presentation of patient data, such as the calculated cardiac output and/or physiological parameters determined by other patient monitoring systems (e.g., electrocardiographic (ECG) monitoring system, a respiratory monitoring system, a blood pressure monitoring system, etc.). In certain embodiments, the display 114 may provide a graph of cardiac output over time (e.g., a cardiac output trend), as well as the current cardiac output value. Alternatively, the patient data (e.g., oxygen saturation and cardiac output) may be displayed on the display 14 of the monitor 12.

Furthermore, as described above, an alarm may be provided in response to determining that the cardiac output value falls outside of a predetermined threshold range. Accordingly, the processor 32, 110 may be configured to determine the threshold range for each patient 22. For example, the clinician may input via control inputs 112 a patient's age, weight, height, gender, or information about the patient's clinical condition that may be relevant to the determination of a threshold range corresponding to normal values for cardiac output. The processor 32, 110 may use the parameters provided by the clinician to determine the threshold range using various algorithms. In the event that the cardiac output parameter falls outside of the threshold range, the monitor 12 or the multi-parameter monitor 92 may provide an alarm, such as an error indication on the display 14, 114, or a beep or other auditory alarm via speaker 16, 116.

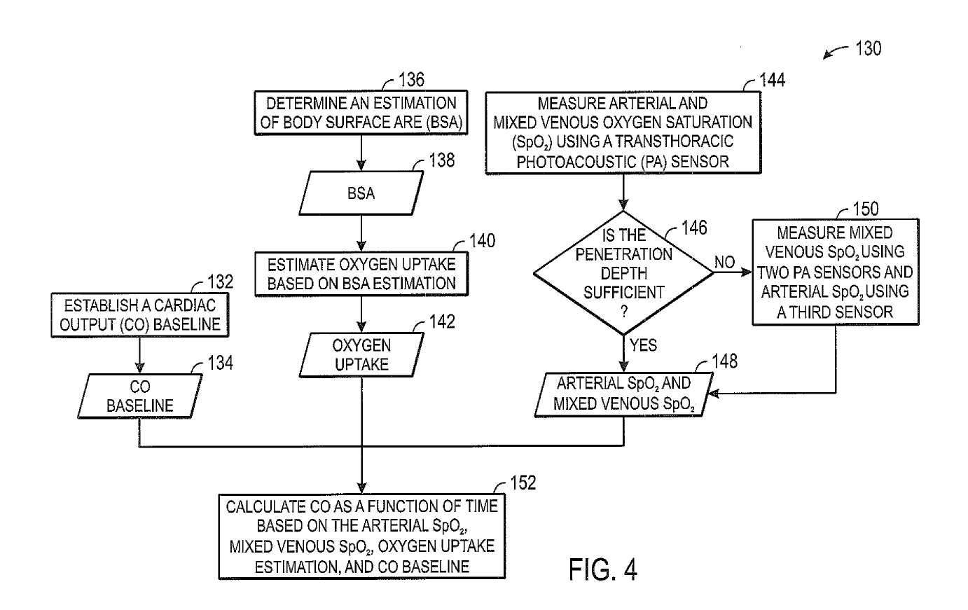

FIG. 4 is a method 130 for calculating and monitoring cardiac output over time (e.g., a cardiac output trend) using one or more photoacoustic sensors 10 and an oxygen uptake estimation using the system 90 of FIG. 3. The method 130 may be performed as an automated procedure by a system, such as the system 90. In addition, certain steps of the method may be performed by a processor, or a processor-based device such as the monitor 12 and/or the multi-parameter monitor 92 that includes instructions (e.g., code) for implementing certain steps of the method 130.

According to an embodiment, the method 130 may include establishing a cardiac output baseline 134 for the patient 22 (block 132). As described above, this may include analyzing blood samples taken from the patient 22 by a clinician to implement the direct Fick method. In other embodiments, establishing the cardiac output baseline 134 may include other known clinical techniques such as the thermodilution technique, echocardiographic technique, or radionuclide imaging technique (block 132). In certain embodiments, an additional system may measure the cardiac output baseline 134, and the monitor 12, 92 may receive, and accordingly establish the cardiac output baseline 134 (block 130) from the additional system or from a clinician via the control inputs 112. As will be described in more detail below, a CO baseline 134 may be used to calibrate a calculation of cardiac output and determine a trending measurement of cardiac output (e.g., cardiac output as a function of time).

The method 130 may also include determining an estimation of body surface area (BSA) 138 for the patient 22 (block 136). In certain embodiments, the processor 32, 110 may determine an estimation of the BSA 138 (block 136) using algorithms and patient parameters inputted by a clinician (e.g., height and weight of the patient 22). The processor 32, 110 may then use the determined BSA 138 to estimate (block 140) the oxygen uptake 142, as described above.

Additionally, the method 130 may include measuring arterial and mixed venous oxygen saturation using a transthoracic photoacoustic sensor 10 (block 144) disposed about the pulmonary artery 96 and the pulmonary vein 98 of the patient 22. As discussed above, it may be desirable to measure the mixed venous oxygen saturation directly from the pulmonary artery 96 and additionally, to measure arterial and mixed venous oxygen saturation using the same sensor 10. However, for certain patients 22, the transthoracic sensor 10 may not generate signals with sufficient penetration depth to accurately measure the arterial and mixed venous oxygen saturation. Accordingly, the method 130 may include performing a signal quality assessment to determine whether the penetration depth of the transthoracic sensor 10 is sufficient (block 146). Specifically, performing a signal quality assessment may include comparing a signal generated using the transthoracic sensor 10 to a signal quality threshold value. For example, determining that the penetration depth of the transthoracic sensor 10 is sufficient may include determining that the signal generated using the transthoracic sensor 10 is above or below the signal quality threshold value. If the penetration depth is sufficient, the processor 32 of the monitor 12 may continue to process signals from the transthoracic sensor 10 to calculate the arterial and mixed venous oxygen saturation 148 of the patient 22.

However, if the penetration depth is not sufficient, the method 130 may include measuring the mixed venous oxygen saturation using multiple (e.g., at least two) photoacoustic sensors 10 and the arterial oxygen saturation using another sensor (e.g., sensor 10) (block 150). Specifically, the processor 32 may send signals to deactivate the light drive 42 of the transthoracic sensor 10 and signals to activate the light drives 42 of the sensor 10 disposed about the jugular artery 100 and the sensor 10 disposed about the femoral artery 102. The processor 32 may then derive the mixed venous saturation 148 based on the signals received from the jugular and femoral sensors 10. Additionally, the processor 32 may activate the light drive 42 of a third photoacoustic sensor 10 disposed about a peripheral artery of the patient 22 to measure the arterial oxygen saturation 148. Alternatively, a pulse oximetry sensor may also be coupled to the monitor 12 and disposed about a peripheral artery of the patient 22 to measure the arterial oxygen saturation 148.

Once the parameters of the Fick equation have been determined, the monitor 12, 92 may calculate cardiac output as a function of time based on the cardiac output baseline 134, the oxygen uptake 142, and the arterial and mixed venous oxygen saturation 148 (block 152). Specifically, the processor 32, 110 may apply various algorithms, such as Equation 3 and 4, to determine cardiac output over time. Additionally, as described with respect to FIG. 2, the cardiac output values may be displayed (block 78) on the display 14, 114 for a clinician to monitor. Furthermore, the processor 32, 110 may be configured to determine a threshold range for normal or acceptable cardiac output values, based at least in part on the patient parameters inputted by a clinician, and provide (block 80) an alarm and/or error indication in response to determining that the cardiac output is outside of the threshold range (e.g., below a low threshold and/or above a high threshold).

In other embodiments, it may be desirable to additionally measure oxygen uptake over time to determine and monitor cardiac output. Additionally, for certain patients 22 (e.g., for patients 22 who are overweight or obese), the body surface area estimate may not yield an accurate oxygen uptake determination. For example, a cardiac output baseline which significantly differs from the cardiac output determination may alert a clinician that the oxygen uptake estimation is inaccurate. As such, the disclosed embodiments provide a system and method for non-invasively monitoring cardiac output via the Fick method using continuous or intermittent measurements of oxygen uptake determined from the inspired and expired air of the patient 22.

With the foregoing in mind, FIG. 5 illustrates a system 170 that may be operable to monitor the cardiac output of the patient 22 using oxygen uptake measurements. The system 170 includes the one or more photoacoustic sensors 10, the monitor 12, and the multi-parameter monitor 92. Again, for illustration purposes the femoral and jugular sensors 10 and the pulse oximetry sensor 106 are coupled to the monitor 12 with dashed lines to indicate that they may not be present in certain embodiments. The system 170 additionally includes a gas analysis device 172, which may be in line with a ventilator 174. In certain embodiments, the gas analysis device 172 may be incorporated into the ventilator 174.

The gas analysis device 172 may be configured to receive and process the inhaled and exhaled gases to calculate oxygen uptake. Specifically, the gas analysis device 172 is may include a mass flow device, an oxygen analyzer, a carbon dioxide analyzer, which may include a pressure sensor, a temperature sensor, viscometer, and/or densitometer, to measure the density, viscosity, and specific heat of the inhaled and exhaled gases. The gas analysis device 172 may include a processor that may apply various algorithms, which may be stored in a suitable computer-readable storage medium, to determine the concentration of oxygen in the inhaled and in the exhaled gases based on the measurements from the mass flow meter and the gas analyzer. Additionally, or alternatively, the gas analysis device 172 may be configured to determine the concentration of carbon dioxide in the inhaled and exhaled gases, as carbon dioxide production may be used in place of oxygen uptake to calculate cardiac output using the Fick method. As the gas analysis device 172 is in line with the ventilator 174, oxygen uptake may be calculated continuously.

However, in other embodiments, the gas analysis device 172 may calculate oxygen uptake intermittently. For example, the patient 22 may not be connected to the ventilator 174. As such, the gas analysis device 172 may include a mask configured to be placed over the nose and mouth of the patient 22 to deliver air and to receive exhaled air. Accordingly, the gas analysis device 172 may analyze each exhaled breath and may calculate oxygen uptake for each breath. While the gas analysis device 172 with the mask does not provide continuous measurements, it may be desirable for certain patients 22 as it may be less invasive.

Accordingly, the multi-parameter monitor 92 or the monitor 12 may be configured to receive the oxygen uptake calculations from the gas analysis device 172 and to use the calculations to determine and monitor the cardiac output of the patient 22. The multi-parameter monitor 92 and the monitor 12 may function as described above. That is, in one embodiment, the multi-parameter monitor 92 may receive the arterial and mixed venous oxygen saturations 148 from the monitor 12, which may be obtained using the one or more photoacoustic sensors 10 and in certain embodiments, the pulse oximetry sensor 106. Upon receiving the parameters of oxygen uptake and arterial and mixed venous oxygen saturation 148, the multi-parameter monitor 92 may then calculate cardiac output as a function of time. Specifically, in certain embodiments, the processor 32, 110 may apply Equation 2 to calculate the arterial and mixed venous oxygen content and then, may use the oxygen content parameters to calculate cardiac output using Equation 1 (e.g., the direct Fick method). The monitor 12, 92 may not utilize a cardiac output baseline 134 to calibrate the cardiac output calculation, as the oxygen uptake calculations may be more accurate than the oxygen uptake 142 determined using the body surface area estimate 138.

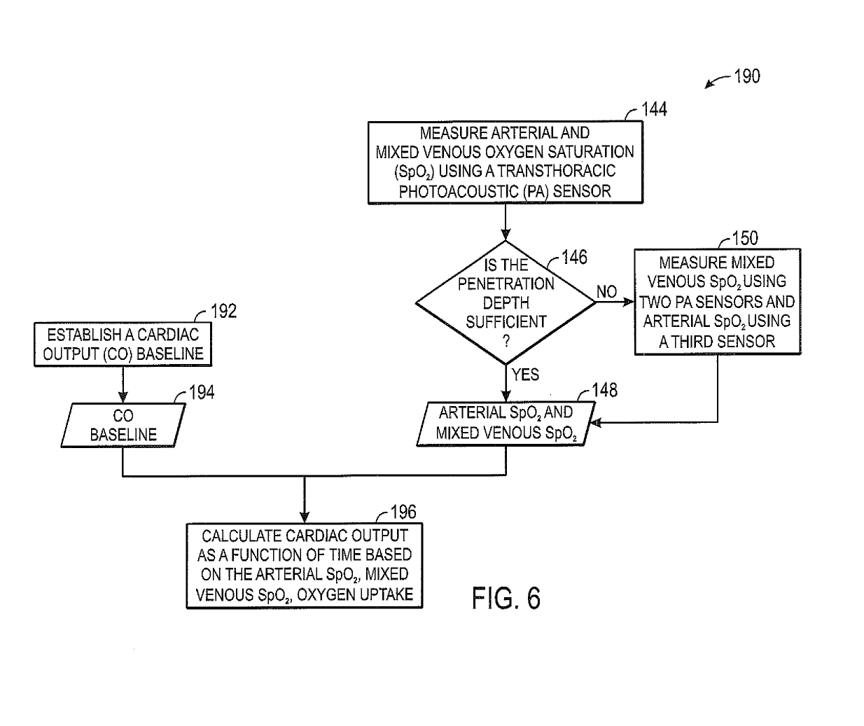

FIG. 6 illustrates a method 190 for calculating and monitoring cardiac output over time using one or more photoacoustic sensors 10 and calculations of oxygen uptake using the gas analysis device 172. The method 190 may be performed as an automated procedure by a system, such as the system 170. In addition, certain steps of the method may be performed by a processor, or a processor-based device such as the monitor 12, the multi-parameter monitor 92, and/or the processor of the gas analysis device 172 that includes instructions for implementing certain steps of the method 190.

Similar to the method 130 described above, the method 190 may include measuring (block 144) arterial and mixed venous oxygen saturation using the transthoracic sensor 10. The method 190 may also include determining (block 146) whether the penetration depth is sufficient to determine the arterial and mixed venous oxygen saturation 148. In response to determining (block 146) that the penetration depth is insufficient, the method may include measuring (block 150) the arterial and mixed venous oxygen saturations using the jugular and femoral sensors 10 and either a third sensor 10 or a pulse oximetry sensor 106 disposed about a peripheral artery of the patient 22.

However, unlike the method 130, the method 190 may include measuring the oxygen uptake 194 using the gas analysis device 172 (block 192). As described above, the gas analysis device 172 may measure oxygen uptake continuously when in line with the ventilator 174, or intermittently for embodiments in which the gas analysis device 172 includes a mask for analyzing each breath of the patient 22. It should be appreciated that the oxygen uptake 194 may differ from the oxygen uptake 142. Accordingly, the multi-parameter monitor 92 may then calculate (block 196) cardiac output as a function of time based on the arterial and mixed venous oxygen saturation 148 and the oxygen uptake 194. In certain embodiments, the multi-parameter monitor 92 may continuously calculate cardiac output using intermittent measurements of oxygen uptake 194 and continuous measurements of oxygen and mixed venous oxygen saturation 148. In one embodiment, the multi-parameter monitor may continuously calculate cardiac output using continuous measurements of oxygen uptake 194 and continuous measurements of oxygen and mixed venous oxygen saturation 148. Additionally, the method 190 may further include displaying (block 78) the calculated cardiac output on the display 114 for a clinician to monitor and review, as well as provide (block 80) an alarm in response to determining that the cardiac output is outside of the predetermined range.

The disclosed embodiments may be interfaced to and controlled by a computer readable storage medium having stored thereon a computer program. The computer readable storage medium may include a plurality of components such as one or more of electronic components, hardware components, and/or computer software components. These components may include one or more computer readable storage media that generally store instructions such as software, firmware and/or assembly language for performing one or more portions of one or more implementations or embodiments of an algorithm as discussed herein. These computer readable storage media are generally non-transitory and/or tangible. Examples of such a computer readable storage medium include a recordable data storage medium of a computer and/or storage device. The computer readable storage media may employ, for example, one or more of a magnetic, electrical, optical, biological, and/or atomic data storage medium. Further, such media may take the form of, for example, floppy disks, magnetic tapes, CD-ROMs, DVD-ROMs, hard disk drives, andor solid-state or electronic memory. Other forms of non-transitory and/or tangible computer readable storage media not list may be employed with the disclosed embodiments.

A number of such components can be combined or divided in an implementation of a system. Further, such components may include a set and/or series of computer instructions written in or implemented with any of a number of programming languages, as will be appreciated by those skilled in the art. In addition, other forms of computer readable media such as a carrier wave may be employed to embody a computer data signal representing a sequence of instructions that when executed by one or more computers causes the one or more computers to perform one or more portions of one or more implementations or embodiments of a sequence.

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Rather, the various embodiments may cover all modifications, equivalents, and alternatives falling within the spirit and scope of the disclosure as defined by the following appended claims.

What is claimed is:

1. A method for non-invasively determining cardiac output, comprising:
determining a mixed venous oxygen saturation value and an arterial oxygen saturation value of a patient, wherein the mixed venous and the arterial oxygen saturation values are based at least in part upon at least one signal generated from at least one photoacoustic spectroscopy sensor;
determining an oxygen uptake of the patient; and
determining the cardiac output of the patient based at least in part upon the mixed venous and the arterial oxygen saturation values and the oxygen uptake using a non-invasive Fick method.

2. The method of claim 1, wherein the at least one signal is generated from a transthoracic photoacoustic spectroscopy sensor.

3. The method of claim 3, comprising:
performing a signal quality assessment on the signal generated from the transthoracic photoacoustic spectroscopy sensor; and
determining the mixed venous oxygen saturation value and the arterial oxygen saturation value of the patient based at least in part upon signals generated from each of two or more photoacoustic spectroscopy sensors if the signal generated from the transthoracic photoacoustic spectroscopy sensor falls below a signal quality threshold.

4. The method of claim 3, wherein the two or more photoacoustic spectroscopy sensors comprise a first photoacoustic spectroscopy sensor disposed about a jugular vein of the patient and a second photoacoustic spectroscopy sensor disposed about a femoral vein of the patient.

5. The method of claim 1, comprising providing an alarm when the cardiac output is outside of a predetermined range.

6. The method of claim 1, wherein determining the oxygen uptake of the patient comprises estimating the oxygen uptake based on a body surface area estimate of the patient.

7. The method of claim 1, wherein determining the oxygen uptake of the patient comprises receiving a calculated oxygen uptake continuously or intermittently from a gas analysis device, wherein the gas analysis device comprises a processing device configured to analyze inhaled and exhaled air of the patient and to calculate the oxygen uptake.

8. The method of claim 1, wherein determining cardiac output comprises determining a cardiac output trend over a period of time.

9. A system to non-invasively determine cardiac output, comprising:
one or more photoacoustic sensors configured to emit light into one or more regions of tissue of a patient, to detect acoustic energy generated in response to the emitted light, and to generate one or more signals based on the detected acoustic energy;
a monitor comprising a processing device configured to:
receive the one or more signals from the one or more photoacoustic sensors;
calculate a mixed venous oxygen saturation value and an arterial oxygen saturation value for the patient based in part upon the one or more received signals;
receive or determine an oxygen uptake of the patient; and
calculate the cardiac output of the patient based at least in part on the mixed venous oxygen saturation value, the arterial oxygen saturation value, and the oxygen uptake using a non-invasive Fick method.

10. The system of claim 9, wherein the processing device of the monitor is configured to:
determine a body surface area estimate of the patient; and
determine an oxygen uptake estimation based at least in part upon the body surface area estimation.

11. The system of claim 10, wherein the processing device of the monitor is configured to:
receive a cardiac output baseline for the patient; and
determine a cardiac output trend over time based upon the cardiac output baseline, the oxygen uptake estimation, the mixed venous oxygen saturation value, and the arterial oxygen saturation value of the patient.

12. The system of claim 9, wherein the processing device of the monitor is configured to receive a calculated oxygen uptake of the patient continuously or intermittently from a gas analysis device, wherein the gas analysis device comprises a processing device configured to analyze inhaled and exhaled air of the patient and to calculate the oxygen uptake.

13. The system of claim 9, wherein the processing device of the monitor is configured to perform a signal quality assessment on the one or more signals from the one or more photoacoustic sensors and to select which of the one or more photoacoustic sensors to use in determining the mixed venous oxygen saturation value and the arterial oxygen saturation value based on the signal quality assessment.

14. The system of claim 13, wherein the one or more photoacoustic sensors comprises a transthoracic photoacoustic sensor, and wherein the signal quality assessment comprises determining whether a penetration depth of the transthoracic photoacoustic sensor is sufficient for measuring the mixed venous oxygen saturation value and the arterial oxygen saturation value of the patient.

15. The system of claim 9, wherein the processing device of the monitor is configured to:
compare the calculated cardiac output of the patient to a predetermined threshold range; and
provide an alarm if the calculated cardiac output is outside of the predetermined threshold range.

16. A patient monitor for non-invasively determining cardiac output, comprising:
a processing device configured to:
receive one or more signals from one or more photoacoustic sensors disposed about a patient;
calculate a mixed venous oxygen saturation value and an arterial oxygen saturation value for the patient based in part upon the one or more received signals;
receive or determine an oxygen uptake of the patient; and
calculate the cardiac output of the patient based in part on the mixed venous oxygen saturation value, the arterial oxygen saturation value, and the oxygen uptake using a non-invasive Fick method.

17. The patient monitor of claim 16, wherein the processing device of the monitor is configured to perform a signal quality assessment on the one or more signals from the one or more photoacoustic sensors and to select which of the one or more photoacoustic sensors to use in determining the mixed venous oxygen saturation value and the arterial oxygen saturation value based on the signal quality assessment.

18. The patient monitor of claim 16, wherein the processing device of the monitor is configured to determine an oxygen uptake estimation of the patient based on a body surface area estimate of the patient.

19. The patient monitor of claim 18, wherein the processing device of the monitor is configured to:
receive a cardiac output baseline for the patient; and
determine a cardiac output trend over time based upon the cardiac output baseline, the oxygen uptake estimation, the mixed venous oxygen saturation value, and the arterial oxygen saturation value of the patient.

20. The patient monitor of claim 16, wherein the processing device of the monitor is configured to:
compare the calculated cardiac output of the patient to a predetermined threshold range; and
provide an alarm if the calculated cardiac output is outside of the predetermined threshold range.
